(19) United States
(12) Patent Application Publication
(10) Pub. No.: US 2026/0109923 A1
(43) Pub. Date: Apr. 23, 2026

(54) METHOD AND APPARATUS FOR PREPARING A TUBULAR STRUCTURE

(71) Applicants: The University Court of the University of Edinburgh, Edinburgh (GB); University of Strathclyde, Glasgow (GB)

(72) Inventors: Ian Holland, Edinburgh (GB); Jamie Davies, Edinburgh (GB); Wenmiao Shu, Glasgow (GB)

(21) Appl. No.: 19/120,553

(22) PCT Filed: Oct. 25, 2023
(86) PCT No.: PCT/GB2023/052793

§ 371 Date: Apr. 11, 2025

(30) Foreign Application Priority Data
Oct. 27, 2022 (GB) 2215946.1

(51) Int. Cl. C12M 3/00; C12M 1/12; C12M 1/36; C12N 5/071

(52) U.S. Cl. CPC C12M 21/08; C12M 23/06; C12M 25/14; C12M 41/48; C12N 5/0697; C12N 2533/54

(57) ABSTRACT

An apparatus (105) for manufacturing a tubular biological structure (165) comprises a tubular member (150) configured to be rotated, wherein the tubular member (150) comprises a tubular wall extending from a first end (151) and a second end (152), wherein the tubular member (150) is configured to receive a biological material (160) on an internal surface (154) thereof, and wherein the tubular member (150) comprises at least one opening (156) through the tubular wall configured to allow egress of excess biological material (160), in use.

### FIELD OF THE INVENTION

The present invention relates to an apparatus and methods for preparing a tubular structure. In particular, but not exclusively, the invention relates to an apparatus and methods for preparing a tubular multilayer biological structure.

### BACKGROUND

Layered tissue is common throughout human anatomy, with notable examples found in cardiac, dermal, intestinal and vascular tissue. Such stratification features a range of different cell types and extracellular matrices to create highly specialised composite structures that imbue tissues with multiple properties and functions. Tissue engineering intended to create anatomically and functionally accurate tissue often makes extensive use of cells suspended in liquid-phase hydrogels that can be deposited and subsequently gelled. When using such an approach to form layered tissue, researchers have few technical options. Extrusion-based 3D bioprinting technologies, the mainstay of the industry, are ill-suited to assembling hydrogels into planar layered structures. In particular, they are unable to attain the requisite deposition resolution and repeatability needed for assembling the multiple thin continuous layers observed in native tissue structures. These technologies also impose constraints on the range of compatible hydrogels with printed liquids requiring rapid gelation to assume the intended 3D spatial form.

Recognising the limitations of 3D bioprinters to manipulate hydrogels into microscale layers, some researchers have attempted to develop improved biofabrication technologies. The sequential, dipping, spraying or direct extrusion of cell-laden hydrogels onto the external surface of cylinders to build up layers is one such strategy. An advantage of this method resides in the formation of a tubular macro-architecture, observed in many native layered tissues such as vascular, intestinal, tracheal and biliary tissue. Furthermore, cutting open layered tubular fabricated tissue allows simple conversion to planar tissue. A limitation of rod-based methods is their requirement to dip or spray cell solutions onto the external surface. This necessitates large volumes of cell-laden hydrogels that are ultimately wasted, and inhibits the cell densities attainable. Removal of the fabricated tissue can also be challenging due to hydrogel shrinkage onto the cylindrical mould. Rotation of the cylinder at high speeds under motor control enables a level of control over the layer thicknesses that is otherwise determined by the viscosity of the liquid hydrogel and the surface properties of the substrate layer. However, this approach limits the density of cells that can be attained in the resulting layer. This means that they are further away from the structure of native tissues, lowering their clinical relevance.

For example, U.S. Pat. No. 10688694B2 (Acevedo et al) discloses a method for the fabrication of multilayer hollow tubes that uses a layer-by-layer rod dipping approach using different biomaterials.

An alternative method is centrifugal casting, where a material is applied to the inside of a closed cylinder that is then rotated. Centrifugal forces push the material, such as hydrogel, to coat the internal surface creating a tube with a layer thickness, typically about 1 mm, determined by the volume added. The technology, however, has received limited attention since the initial pioneering work by Mironov et al.

Mironov et al (“Fabrication of tubular tissue constructs by centrifugal casting of cells suspended in an in situ crosslinkable hyaluronan-gelatin hydrogel”; Biomaterials 26 (2005) 7628-7635) discloses the use of centrifugal casting to produce tubular biological constructs made from crosslinkable hyaluronan-based (HA) synthetic extracellular matrix (sECM).

U.S. Pat. No. 6,969,480 (Dalton et al) discloses hollow structures manufactured with a rotational spinning technique. In this document, phase separation of soluble solutions or emulsions was induced within a filled mold as it was being rotated about one of its axis, and the density difference between phases results in sediment at the inner lumen of the mold under centrifugal forces.

EP2755599B1 discloses engineered tissues and organs comprising one or more layers of muscle, the engineered tissue or organ consisting essentially of cellular material, provided that the engineered tissue or organ is implantable in a vertebrate subject and not a vascular tube.

However, the centrifugal casting approach is typically ill suited to the preparation of multi-layered biological structures because the additional of variable hydrogel volumes forms inconsistent thicknesses. In addition, biological material such as collagen has typically proved difficult to manipulate using existing biofabrication technologies due to a combination of low viscosity and extended thermal gelation time requiring a high level of self-support and temperature control when transitioning from a liquid to a gel.

It is an object of the invention to address and/or mitigate one or more problems associated with the prior art.

It is an object of the invention to provide an improved method of manufacturing a tubular biological structure, e.g. a multi-layered tubular biological structure.

### SUMMARY

According to a first aspect, there is provided an apparatus for manufacturing a tubular biological structure, the apparatus comprising:
a tubular member configured to be rotated,
wherein the tubular member comprises a tubular wall extending between a first or proximal end and a second or distal end,
wherein the tubular member is configured to receive a biological material on an internal surface thereof, and
wherein the tubular member comprises at least one opening through the tubular wall configured to allow egress of excess biological material, in use.

The apparatus may comprise a rotatable support configured to be rotated. The tubular member may be configured to be mounted on or attached to the rotatable support.

The tubular member, e.g. tubular wall, may define an external surface and an internal surface.

Typically, the tubular member may define an open tube. The first end of the tubular member may define a first open end. The second end of the tubular member may define a second open end. The first end may be provided opposite the second end. The first end may be or may define a proximal end. The second end may be or may define a distal end.

Advantageously, the tubular member may have a substantially constant diameter between the first end and the second end. The tubular member may have or may define a substantially constant or continuous circular cross-section between the first end and the second end. By such provision, upon rotation, an even thickness of biological material on the internal surface of the tubular member may be achieved.

The apparatus may comprise a motor capable of actuating rotation of the tubular member.

The support may be connected to or may be actuated by a motor. The support may be rotated by actuating the motor. Thus, when the tubular member is mounted on or attached to the support, the tubular member may be rotated via the rotatable support by actuating the motor.

The support may be configured to engage with the tubular member, e.g. tubular wall.

The support and the tubular member may have complimentary features.

The support may comprise a tubular or cylindrical portion.

The cylindrical portion of the support may be configured to fit within an end, e.g. a/the first or proximal end, of the tubular member. An external surface of the cylindrical portion of the support may be configured to engage with an internal surface of an end, e.g. a/the first or proximal end, of the tubular member.

The tubular portion of the support may be configured to fit on an external surface of the tubular member. An internal surface of the tubular portion of the support may be configured to engage with an external surface of an end, e.g. a/the first end, of the tubular member.

The support and the tubular member may be configured to engage with the tubular member, e.g. first or proximal end thereof, to form a waterproof seal. There may be provided one or more seals to provide a sealed engagement between the support and the tubular member.

In use, the support may extend substantially horizontally. The support may be configured to cause rotation of the tubular member on a substantially horizontal axis. This may help achieve a consistent layer thickness of the biological material.

The apparatus may comprise a blocking element. The blocking element may be configured to prevent egress of the biological material, in use, at the second or distal end.

The blocking element may typically be provided at an end, e.g. at the second or distal end, of the tubular member.

The blocking element may be separate and/or distinct from the tubular member.

The blocking element may be configured to provide a sealed engagement with the tubular member, e.g. at a/the second or distal end thereof.

The blocking element may be configured to engage with an internal surface and/or with an external surface, of the tubular member. Typically, the blocking element may be configured to engage with an internal surface of the tubular member.

The apparatus may be configured to allow the delivery of the biological material within the tubular member, e.g. on an internal surface of the tubular member, e.g. tubular wall. In an embodiment, the blocking element may be configured to allow the delivery of the biological material within the tubular member, e.g. on an internal surface of the tubular member, e.g. tubular wall.

The blocking element may comprise an opening therein. The blocking element may comprise an opening near a central region thereof. By such provision, the opening may allow the delivery of the biological material without compromising the ability of the blocking element to prevent egress of the biological material upon rotation to the tubular member.

The blocking element may comprise or may be a stopper.

The stopper may be made of a resilient or compressible material, e.g. foam or rubber. By such provision, the opening may not compromise the stopper's ability to prevent egress of the biological material, whilst allowing an object, e.g. a delivery device such as a syringe, to be inserted through the opening to permit delivery of the biological material.

The blocking element may comprise or may be a tubular blocking element. The tubular blocking element may be rigid. The tubular blocking element may be made of a rigid material such as plastic, metal, or glass.

The tubular blocking element may be configured to engage with an end, e.g. second or distal end, of the tubular member.

The tubular blocking element may be partially extend from the second or distal end into the tubular member.

The blocking element, or part of the blocking element, may be integral to the tubular member.

The blocking member may comprise or may define a ridge or shoulder which may typically extend radially inwards at or near end end, e.g. second or distal end, of the tubular member.

The tubular member, e.g. second or distal end thereof, may comprise or may define a/the ridge or shoulder which may typically extend radially inwards. Thus, the blocking element may define an opening having a dimension, e.g. inner diameter, less than a dimension, e.g. inner diameter, of the tubular member. By such prvision, in use, the blocking member, e.g. ridge or shoulder, may allow the delivery of the biological material within the tubular member, and may prevent egress of the biological material upon rotation to the tubular member.

The blocking element may comprise one or more of the above features in combination.

In an embodiment, the blocking element may comprise:
a ridge or shoulder which may typically extend radially inwards at or near end end, e.g. second or distal end, of the tubular member; and
a tubular blocking element which may be partially extend from the second or distal end into the tubular member.

The ridge or shoulder, and the tubular blocking element, may be integral or one-piece. Alternatively, the tubular blocking element may be separate from the ridge or shoulder. The tubular blocking element may be configured to engage with the ridge or shoulder.

The tubular member may define an annulus extending from the second or distal end between an inner surface of the tubular member and the tubular blocking member. This may help prevent egress of the biological material upon rotation to the tubular member.

The tubular member comprises at least one opening configured to allow egress of excess biological material, in use. The at least one opening may extend through the wall of the tubular member from an internal surface to an external surface thereof.

The at least one opening may define or may act as at least one drain hole.

The tubular member may comprise a plurality of openings configured to allow egress of excess biological material, in use.

Advantageously, the provision of at least one opening, e.g. a plurality of openings, in the tubular member, may allow precise control over the thickness of each layer of biological material.

Advantageously also, the provision of at least one opening, e.g. a plurality of openings, in the tubular member, may allow purging of any gelation/crosslinking agent that may need to be added to the biological material to initiate gelling and/or crosslinking thereof.

For example, when the biological material is a hydrogel, e.g. a low-viscosity hydrogel, formation of a layer of the biological material may be followed by addition of a gelation and/or crosslinking agent. In use, following addition of the gelation and/or crosslinking agent, the tubular member may be rotated so as to distribute, e.g. substantially evenly distribute, the gelation and/or crosslinking agent over the biological material. The provision of at least one opening in the tubular member provides a route for any excess gelling and/or crosslinking agent to escape, thus avoiding or reducing the risk of unreacted residual gelling and/or crosslinking agent on the surface of the biological material before a fresh layer of the biological material, e.g. hydrogel, is added, thereby avoiding unwanted premature crosslinking and/or gelling thereof.

However, it will be appreciated that gelling and/or crosslinking may, depending on the nature of the biological material, not require addition of a separate gelation and/or crosslinking agent. In such instance, the provision of at least one opening, e.g. a plurality of openings, in the tubular member, may still allow precise control over the thickness of each layer of biological material. The openings may be circumferentially disposed on the tubular member, e.g. tubular wall. The openings may be located in a plane substantially perpendicular to the longitudinal axis of the tubular member. The openings may be located at substantially the same distance from an end, e.g. second end, of the tubular member.

The positioning of the openings may allow the egress of excess biological material to be confined to a particular location, and to be collected upon exit. This may be particularly useful if the material being used is of a high value and can be reused, as it may reduce waste of the biological material. This is particularly true compared to other approaches forming layers on an external surface of a rotating rod.

The openings may be symmetrically disposed around a circumference of the tubular member. This may help evenly distribute the flow of any excess biological material around the circumference of the tubular member, thus providing a consistent layer thickness on an internal surface of the tubular member.

The size of the openings may be substantially the same. This may further help evenly distribute the flow of any excess biological material around the circumference of the tubular member, thus providing a consistent layer thickness on an internal surface of the tubular member.

There may be provided N openings, disposed circumferentially at about 360°/N relative to each other. For example, there may be provided 3 openings disposed at about 120° relative to each other. There may be provided 4 openings disposed at about 90° relative to each other. There may be provided 5 openings disposed at about 72° relative to each other. There may be provided 6 openings disposed at about 60° relative to each other.

There may be provided a plurality of sets of openings, the openings of each set being symmetrically disposed around a circumference of the tubular member.

There may be provided a first set of openings being symmetrically disposed around a circumference of the tubular member, the first set of openings located near a second end of the tubular member and placed at a first distance from the second end of the tubular member. The first set of openings may include N₁ openings disposed circumferentially at about 360°/N₁ relative to each other.

There may be provided a second set of openings being symmetrically disposed around a circumference of the tubular member, the second set of openings located near a first end of the tubular member and placed at a second distance from the second end of the tubular member. The second set of openings may include N₂ openings disposed circumferentially at about 360°/N₂ relative to each other.

Typically, the at least one opening, e.g. the first set of openings and/or the second set of openings, may be substantially circular. This may help egress of the biological material.

The at least one opening, e.g. the first set of openings and/or the second set of openings, may comprise a chamfer on the internal surface of the tubular member, e.g. tubular wall. This may help or may promote egress of the biological material and may help achieve a consistent thickness of biological material.

The number of sets of openings may depend on the location where the biological material is intended to be fed or delivered.

If the biological material is intended to be delivered near an end, e.g. near the first or proximal end, of the tubular member, there may be provided one set of openings. The openings may be provided near the second or distal end of the tubular member. By such provision, upon rotation, the biological material may be coated on an internal surface of the tubular member at least between the delivery location and the set of openings.

If the biological material is intended to be delivered near an opposite end, e.g. near the second or distal end, of the tubular member, there may be provided one set of openings. The openings may be provided near the first or proximal end of the tubular member. By such provision, upon rotation, the biological material may be coated on an internal surface of the tubular member at least between the delivery location and the set of openings. This configuration may allow the biological material to be delivered or dispensed near the second or distal end. Advantageously, this may minimise the insertion of a delivery device, e.g. a syringe or nozzle, towards the first or proximal end of the tubular member. In other words, this may help reduce the overlap between the tubular member and the delivery device, which may in turn help reduce the risk or contact between the tubular member and the delivery device upon rotation.

If the biological material is intended to be delivered away from either end of, e.g. near a central region of, the tubular member, there may be provided either one set of openings near the first end or the second end of the tubular member, or a plurality of sets of openings, e.g. two sets of openings. A first set of openings may be provided near the first end and a second set of openings may be provided near the second end. By such provision, upon rotation, the biological material may be coated on an internal surface of the tubular member at least between the first set of openings and the second set of openings.

An advantage of the present invention is the ability to form multiple layers of the biological material when using a low viscosity material such as a hydrogel. These types of hydrogels are useful from a tissue engineering and biological study perspective, because they often mimic the properties of native tissues. Collagen is an example of such material. Unfortunately, these materials are often difficult to form into biologically relevant structures (such as microscale multilayers) using existing technologies as they are unable to retain their 3D shape in the transition from the liquid to the gelled form. The present invention allows the use of low viscosity biological materials such as hydrogels to form multiple layer structures that mimic the macro and microscale architectures of native tissues.

The biological material may comprise or may be a hydrogel. The biological material may comprise an alginate, collagen, or the like. It will be understood that the term “biological material” will be herein understood in the context of the use for the tubular structure in biological applications. However, the biological material may in some instances comprise synthetic materials suitable for biological applications, e.g. polyether homo-or co-polymers such as pluronic® F-127.

The biological material may comprise a cellular material, e.g. cells. Thus, the present apparatus and methods may allow to preparation of multi-layered cellular structures.

The apparatus may comprise of may be coupled to a first control unit configured to control the rotation of the tubular member. The control unit may be configured to control the actuation of the rotatable support and/or of the motor.

The first control unit may be configured to control the direction of rotation.

The first control unit may be configured to control the speed of rotation. Typically the first control unit may be configured to control and/or adjust the speed of rotation to a speed in the range of about 1000-15000 rpm, e.g. about 4000-10000 rpm.

The apparatus may comprise of may be coupled to a second unit configured to control the delivery of the biological material.

The first control unit and the second control unit may be the same or may be different.

The apparatus, e.g. first and/or second control unit, may be manually actuated or operated.

The apparatus, e.g. first and/or second control unit, may be automatically actuated or operated, e.g. by a computer.

The present invention offers a cost-effective approach to biological tissue engineering, particularly compared to bioprinting equipment which is sometimes a financial barrier to researchers seeking to biofabricate tissue in the laboratory. The speed of layered tissue fabrication using the present methodology presents a further advantage to researchers, especially in comparison to technologies such as cell-sheet engineering which require extended maturation times.

According to a second aspect, there is provided a method of manufacturing a tubular biological structure, the method comprising:
(i) providing an apparatus comprising a tubular member configured to be rotated, wherein the tubular member comprises a tubular wall extending from a first end and a second end, wherein the tubular member comprises at least one opening through the tubular wall configured to allow egress of excess biological material, in use;
(ii) feeding a first amount of a biological material on an internal surface of the tubular member; and
(iii) rotating the tubular member.

The tubular member may be a tubular member as described in relation to the first aspect.

The apparatus may be an apparatus as described in relation to the first aspect.

The method may comprise providing the tubular member on a rotatable support configured to be rotated. The method may comprise mounting the tubular member on the support.

The method may comprise mounting the tubular member on the support such that the tubular member extends substantially horizontally.

The method may comprise feeding a predetermined amount of the biological material. It will be appreciated that the amount of the biological material fed into the tubular member may depend on the size of the tubular member, on the desired thickness of the/each layer of biological material, and/or on the speed of rotation intended to be applied. Although the thickness of each layer is primarily controlled by the speed of rotation and the viscosity of the material, it will be understood that the amount of material fed into the tubular member should be sufficient to generate a complete layer of the material on an internal surface of the tubular member. Typically, the amount, e.g. volume of material, should be sufficient to induce rimming flow on an internal surface of the tubular member, and any excess material may exit via the at least one opening, e.g. drain holes. Thus, it will be appreciated that the minimal amount of material required may depend on the size of the tubular member, and the desired thickness of each layer. Typically, the method may comprise feeding about 0.002-4 ml, e.g. about 0.01-1 ml of biological material.

The method may comprise feeding the biological material via a/the opening in the stopper at the second end of the tubular member.

The method may comprise rotating the rotatable support, thus causing rotation of the tubular member.

The method may comprise forming a layer, e.g. a first layer, of the biological material on an internal surface of the tubular member, e.g. tubular wall.

The method may comprise actuating a motor coupled to the support.

The method may comprise rotating the tubular member on a substantially horizontal axis. This may help achieve a consistent layer thickness of the biological material.

The method may comprise controlling the speed of rotation of the rotatable support and/or of tubular member. This may help achieve a desired layer thickness of the biological material.

The method may comprise rotating the tubular member at a speed of about 1000-15000 rpm, e.g. about 4000-10000 rpm.

Step (ii) may be carried out prior to step (iii). In other words, the first amount of biological material may be fed into the tubular member prior to rotating the tubular member. In such instance, step (ii) may be carried out whilst the tubular member is static and/or not rotating. This may minimise any risk of spillage and/or damage during the feeding step.

Steps (ii) and (iii) may be carried out concomitantly. In such instance, the first amount of biological material may be fed into the tubular member whilst rotating the tubular member. In such instance, step (ii) may be carried out whilst the tubular member is already being rotated. This may improve the homogeneity of the layer thickness within the tubular member.

The method may comprise maintaining the same speed of rotation during step (ii).

Alternatively, the method may comprise feeding the first amount of biological under a first speed of rotation, and subsequently setting a second speed of rotation, e.g. increasing the speed of rotation, to form a respective layer of the material on an internal surface of the tubular member.

The method may comprise allowing egress of excess biological material, during rotation, via the at least one opening, e.g. via the plurality of openings, e.g. drain holes.

The method may comprise forming a layer of the biological material on an internal surface of the tubular member, e.g. tubular wall.

The method may comprise forming a layer of the biological material between a first end of the tubular member and the at least one opening, e.g. a first set of openings.

The method may comprise delivering a predetermined amount of biological material near a first end the tubular member. In such instance, there may be provided one set of openings provided near the second end of the tubular member. The method may comprise forming a layer of the biological material between the first end and the set of openings circumferentially located near the second end.

The method may comprise delivering a predetermined amount of biological material away from either end of the tubular member, e.g. near a central region thereof. In such instance, there may be provided one set of openings provided near the second end of the tubular member, or there may be provided a plurality of sets of openings, e.g. two sets of openings. A first set of openings may be provided near the first end and a second set of openings may be provided near the second end. The method may comprise forming a layer of the biological material between the first end and the set of openings circumferentially located near the second end, or between the first set of openings and the second set of openings.

The method may further comprise:
(iv) adding or feeding a first amount of gelation/crosslinking agent inside the tubular member. By such provision, the gelation/crosslinking agent may initiate gelation and/or crosslinking of the first amount or first layer of biological material.

It will be appreciated that certain biological materials, e.g. alginates, may require the addition of a crosslinking or gelling agent to gel or crosslink. However, other types of biological materials, e.g. collagen or agarose, may not require addition of a crosslinking or gelling agent to gel or crosslink, and therefore may not require this additional step after the formation of each discrete layer of biological material.

The method may comprise feeding the gelation and/or crosslinking agent via a/the opening in the stopper at the second end of the tubular member.

The method may comprise:
(v) rotating the tubular member, e.g. at a speed of about 1000-15000 rpm, e.g. about 4000-10000 rpm.

Preferably, the method may comprise adding or feeding the first amount of gelation/crosslinking agent inside the tubular member during rotation of the tubular member. The method may comprise performing steps (iv) and (v) concomitantly. Advantageously, this may prevent or reduce the risk of local or uneven/heterogeneous crosslinking upon addition of the gelation/crosslinking agent.

In other words, whilst the biological material may be fed either before or during rotation of the tubular member, any addition of a gelation/crosslinking agent may preferably be carried out during rotation of the tubular member in order to increase the homogeneity of the crosslinking process.

The method may comprise allowing egress of excess gelling and/or crosslinking agent, during rotation, via the at least one opening, e.g. via the plurality of openings, e.g. drain holes.

Advantageously, the method may allow substantially even distribution of the gelation and/or crosslinking agent over the biological material. Advantageously also, the method may allow flushing or egress of any excess gelling or crosslinking material from the surface of the first layer of biological material. This may avoid or reduce the risk of unreacted residual gelling and/or crosslinking agent on the surface of the first layer biological material before a fresh layer of the biological material, e.g. hydrogel, is added, thus avoiding unwanted premature crosslinking and/or gelling thereof.

Thus, the method may comprise forming a first gelled or crosslinked layer of the biological material on an internal surface of the tubular member.

The method may comprise actuating crosslinking, e.g. by applying a stimulus, such as irradiation (e.g. UV light) or temperature.

The method may comprise forming a second layer of a second biological material on the first layer. The second biological material and the first biological material may be the same or may be different.

The method may comprise feeding a second amount of a second biological material on an internal surface of the first layer; and
rotating the tubular member.

Feeding the second amount of the second biological material may be carried out either prior to or concomitantly with rotating the tubular member.

The method may comprise adding or feeding a second amount of gelation/crosslinking agent inside the tubular member, preferably whilst rotating the tubular member. By such provision, the gelation/crosslinking agent may initiate gelation and/or crosslinking of the second amount or second layer of biological material.

The method may comprise reiterating this process to form a multi-layer biological structure on an internal surface of the tubular member.

The method may comprise actuating or operating the apparatus, e.g. control unit, manually.

The method may comprise actuating or operating the apparatus, e.g. control unit, automatically.

The method may comprise disconnecting the tubular member from the rotatable support.

The method may comprise removing the stopper.

The method may comprise removing the biological structure from the tubular member.

The method may comprise cutting the structure, e.g. longitudinally, so as to obtain a planar biological structure.

Thus, whilst the initial structure made using the present apparatus and method may be in the form of a tubular structure, the final structure may not be tubular, e.g. may be planar.

According to a third aspect, there is provided a method of manufacturing a tubular multi-layered biological structure, the method comprising:
(i) providing an apparatus comprising a tubular member configured to be rotated, wherein the tubular member comprises a tubular wall extending from a first end and a second end, wherein the tubular member comprises at least one opening through the tubular wall configured to allow egress of excess biological material, in use;
(ii) feeding a first amount of a biological material on an internal surface of the tubular member;
(iii) rotating the tubular member to form a first layer;
(iv) optionally adding or feeding a first amount of gelation/crosslinking agent inside the tubular member to cause gelling and/or crosslinking of the first layer; and rotating the tubular member;
(v) repeating steps (ii)-(vi) to form one or more additional layers.

According to a fourth aspect, there is provided a biological structure obtained or obtainable by the method according to the second aspect or the third aspect.

The biological structure may be tubular.

The biological structure may be planar, e.g. after cutting the tubular structure.

The features described in relation to any aspect of the invention may equally apply to any other aspect and, merely for brevity, are not repeated. For example, features described in relation to the apparatus can apply in relation to methods, and vice versa.

### BRIEF DESCRIPTION OF DRAWINGS

Embodiments of the invention are described with reference to the accompanying drawings, which show:

FIGS. 1-5(b) an apparatus for manufacturing a tubular biological structure, according to a first embodiment;

FIG. 6 multilayer alginate-based hydrogel tubes of different internal diameters, made using the apparatus of FIGS. 1-5(b);

FIG. 7 Bright field microscopy image of a tube wall of a tube of FIG. 6, showing discrete alginate layers, the bottom-right scale bar being 500 μm;

FIG. 8(a)-8(e) Bright field microscopy images of tube walls formed using alginate 1% (w/v) at various rotational speeds, and (e) graph showing the relationship between rotation speed and layer thickness;

FIG. 9(a)-9(e) Bright field microscopy images of tube walls formed using alginate compositions at various concentrations (w/v) a) 4%, b) 2%, c) 1% and d) 0.5%, and (e) graph showing the relationship between concentration and layer thickness;

FIG. 10(a)-10(e) Combined bright field and fluorescence microscopy images of tube walls formed using HEK cells encapsulated into 1% (w/v) alginate showing live (green) and dead (red) cells at a) day 1, b) day 4, c) day 7 and d) day 10, and (e) graph representing cell viability;

FIG. 11(a)-11(d) Combined bright field and fluorescence microscopy images of tube walls showing position of prelabelled red and green high density HEK293 cells in adjacent patterned layers formed using 1% w/v alginate;

FIG. 11(e)-11(f) Combined bright field and fluorescence microscopy images of tube walls showing position of prelabelled red and green high density HEK293 cells layers formed using 1% w/v alginate with either with 5 (FIG. 11(e)) or 10 (FIG. 11(f)) acellular layers therebetween;

FIG. 12(a)-12(i) investigation of tubular structures made using human vascular smooth muscle cells (hSMCS) encapsulated in collagen hydrogel, showing (a) macroscale collagen tube architecture with an opaque appearance; (b) microscale acellular collagen layer formation; (c to f) hSMC viability in collagen with live (green) and dead (red) cells at 1, 4, 7 and 10 days post biofabrication; (g) hSMC orientation 4 days after stratification with yellow arrow indicating circumferential direction; (h and i) confocal stacks showing hSMC positioning with 20 (h) and 10 (i) acellular layer spacing therebetween;

FIG. 13 Distances between the human smooth muscle cell layers caused by a set number of acellular layers in between the 2 populations. These correspond to the double vertical lines in FIG. 12(h) and 12(i);

FIG. 14(a)-14(e) Bright field microscopy images of tube walls formed using agarose 1% (w/v) at various rotational speeds, and (e) graph showing the relationship between rotation speed and layer thickness;

FIG. 15(a)-15(e) Bright field microscopy images of tube walls formed using agarose compositions at various concentrations (w/v) a) 8%, b) 4%, c) 2% and d) 1%, and (e) graph showing the relationship between concentration and layer thickness;

FIG. 16: an apparatus for manufacturing a tubular biological structure, according to another embodiment;

FIG. 17: a comparison of the layer thickness obtained using a manual approach vs an automated approach;

FIG. 18: a comparison of the cell viability measured using a manual approach vs an automated approach.

### DETAILED DESCRIPTION

In the present disclosure, reference is made to a number of terms, which have the meanings provided below, unless a context indicates to the contrary. The nomenclature used herein for defining compounds, in particular the compounds according to the invention, is in general based on the rules of the IUPAC organisation for chemical compounds, specifically the “IUPAC Compendium of Chemical Terminology (Gold Book)”. For the avoidance of doubt, if a rule of the IUPAC organisation is in conflict with a definition provided herein, the definition herein is to prevail. Furthermore, if a compound structure is in conflict with the name provided for the structure, the structure is to prevail.

The term “comprising” or variants thereof is to be understood herein to imply the inclusion of a stated element, integer or step, or group of elements, integers or steps, but not the exclusion of any other element, integer or step, or group of elements, integers or steps.

The term “consisting” or variants thereof is to be understood to imply the inclusion of a stated element, integer or step, or group of elements, integers or steps, and the exclusion of any other element, integer or step or group of elements, integers or steps. The term “about” herein, when qualifying a number or value, is used to refer to values that lie within ±5% of the value specified. For example, if a temperature is specified to be about 5 to about 13° C., temperatures of 4.75 to 13.65° C. are included. Reference to physical states of matter (such as liquid or solid) refer to the matter's state at 25° C. and atmospheric pressure unless the context dictates otherwise.

FIGS. 1-5(b) illustrate an apparatus 105 for manufacturing a tubular biological structure according to a first embodiment, and an associated method of manufacturing a tubular biological structure.

As shown in FIG. 1, the apparatus 105 comprises a motor 110 configured to actuate a rotatable support 120 in the form of a cylinder.

The tubular member 150 defines an open tube having a circular cross-section.

The tubular member 150 has a first end 151 configured to engage with the support 120. The tubular member 150 has a second end 152 facing opposite the support 120.

The tubular member 150 has an external surface 153 on an outer side thereof, and an internal surface 154 on an inner side thereof.

In this embodiment, the support 120 is a cylinder configured to sealably engage with an internal surface 154 of the tubular member 150.

The apparatus 105 includes a stopper 155 configured to fit within the second end 152 of the tube 150 and configured to sealably engage with its internal surface 154, so as to prevent egress of the biological material, in use, at the second end 152.

The stopper 155 is configured to allow the delivery of a biological material 160 within the tubular member 150, typically on an internal surface 154 thereof.

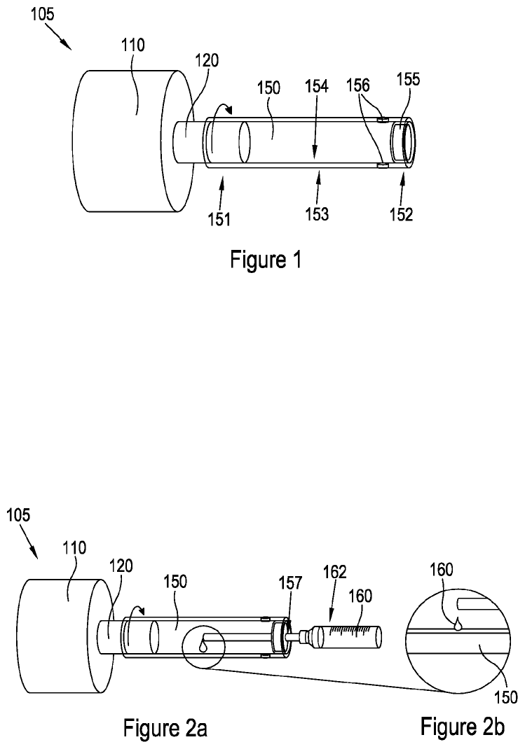

As best shown in FIG. 2(a), the stopper 155 has an opening 157, e.g. a slot or a hole, near a central region thereof. By such provision, the opening 157 allows the delivery of the biological material 160 via a delivery device 162, in this embodiment a syringe, without compromising the ability of the stopper 155 to prevent egress of the biological material 160 upon rotation to the tubular member 150.

The tubular member 150 includes openings 156, in this embodiment two openings, provided near the second end 152 of the tube 150, and configured to allow egress of excess biological material, in use, as will be described later.

FIG. 2 shows a first step of feeding a first amount, typically between 2 μl and 4 ml, of a biological material 160 on an internal surface 154 of the tubular member 150. As explained above, the stopper 155 has an opening 157, e.g. a slot or a hole, near a central region thereof. By such provision, the opening 157 allows the delivery of the biological material 160 via a delivery device 162, in this embodiment a syringe, without compromising the ability of the stopper 155 to prevent egress of the biological material 160 upon rotation to the tubular member 150. As shown in FIG. 2(b), in this embodiment, a predetermined volume of the biological material 160 is delivered near a central region of the tubular member 150. However, as mentioned above, in other embodiments, the biological material 160 might be delivered in a different region of the tubular member 150, for example near an end of the tube 150 opposite stopper 155 and/or openings 156.

As shown by the rotational arrow in FIGS. 1-3, in this embodiment, the tubular member is rotated in a continuous manner. However, as explained above, in other embodiments, the biological material 160 might be delivered under static conditions, and the tubular member 150 might be rotated subsequent to the feeding step.

The delivery device 162 is then removed.

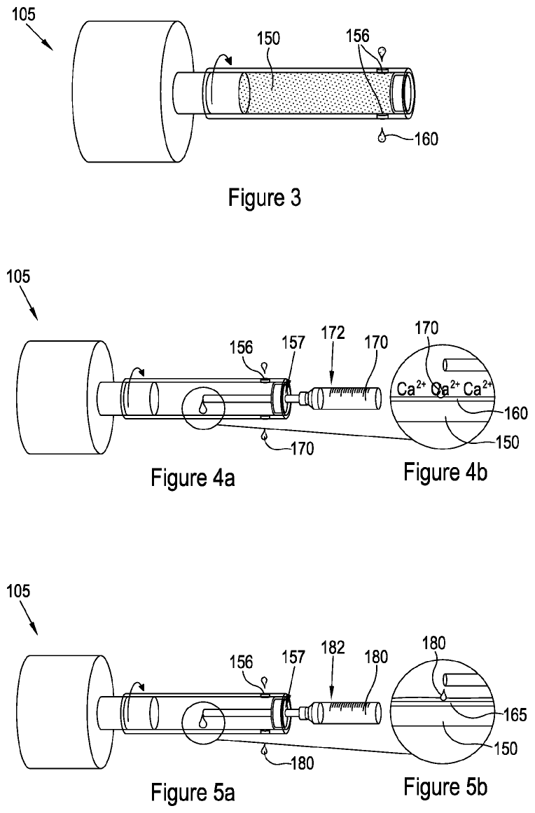

As shown in FIG. 3, the tubular member 150 is rotated at a predetermined rotation speed. Advantageously, the provision of openings 156 in the tubular member 150 allows excess biological material 160 to be expelled from the tube 150, which allows precise control over the thickness of the layer of biological material 160 being formed on the internal surface 154 of the tube 150.

In this embodiment, there are two openings 156 which are disposed diametrically opposite each other. However, it will be appreciated that there may be any number N of openings, preferably disposed circumferentially in a symmetrical fashion, e.g. at an angle of about 360°/N relative to each other.

In this embodiment, the openings 156 are substantially circular, and also have a chamfer on the internal surface 154 of the tubular member 150. This may help or may promote egress of the biological material and may help achieve a consistent thickness of biological material.

Advantageously, the positioning of the openings 156 allows the egress of excess biological material 160 to be confined to a particular location, and to be collected upon exit. This may be particularly useful if the material being used is of a high value and can be reused, as it may reduce waste of the biological material 160. This is particularly true compared to other approaches forming layers on an external surface of a rotating rod.

If the biological material 160 required a gelling or crosslinking agent to be added to gel or crosslink the material between successive layers, e.g. for a hydrogel such as an alginate, then the method comprises a crosslinking step, as illustrated in FIG. 4. In this step, a first amount of a crosslinking or gelling agent 170 (in this embodiment 100 mM CaCl) is fed on an internal surface of the first layer of material 160, via a delivery device 172, in this embodiment a syringe, which is inserted through the stopper 155 via the stopper opening 157. As shown in FIG. 4(b), in this embodiment, a predetermined volume, typically between 2 μl to 4 ml, of the crosslinking agent 170 is delivered near a central region of the tubular member 150. The tube 150 is rotated so as to coat the crosslinking or gelling agent 170 on the surface of the biological (alginate) material 160 and cause crosslinking or gelling thereof. Addition of the first amount of gelation/crosslinking agent 170 inside the tubular member 150 is performed during rotation of the tubular member 150. Advantageously, this may promote homogeneous crosslinking of the biological material 160.

Advantageously also, the provision of the openings 156 allows purging of any excess gelation/crosslinking agent 170, thus avoiding or reducing the risk of unreacted residual gelling and/or crosslinking agent 170 on the surface of the biological material 160 before a fresh layer of biological material is added, thereby avoiding unwanted premature crosslinking and/or gelling thereof.

This step thus creates a first layer 165 of crosslinked or gelled biological material. To build additional layers, the same process is repeated, as illustrated in FIG. 5, in which a second biological material 180 is fed via delivery device 182 on an internal surface of the first layer 165, and the tubular member 150 is rotated so as to create a second layer.

It will be appreciated that, depending on the required application and biological structure desired, the second material 180 (and any subsequent material used for additional layers) may be either the same as, or different from, the first material 160.

An advantage of the present invention is the ability to form multiple layers of a biological material when using a low viscosity material such as a hydrogel. These types of hydrogels are useful from a tissue engineering and biological study perspective, because they often mimic the properties of native tissues. Collagen is an example of such material. Unfortunately, these materials are often difficult to form into biologically relevant structures (such as microscale multilayers) using existing technologies as they are unable to retain their 3D shape in the transition from the liquid to the gelled form. The present invention allows the use of low viscosity biological materials such as hydrogels to form multiple layer structures that mimic the macro and microscale architectures of native tissues.

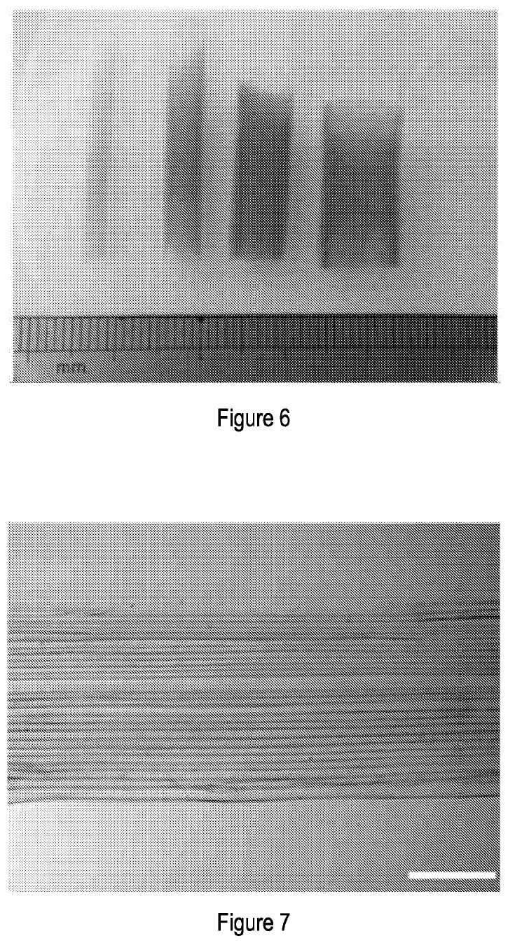

FIG. 6 shows examples of multilayer alginate-based hydrogel tubes formed using the method described above in FIGS. 1-5(b), using tubes 150 of different diameters, in this case 3 mm, 5 mm, 7 mm, and 10 mm, respectively.

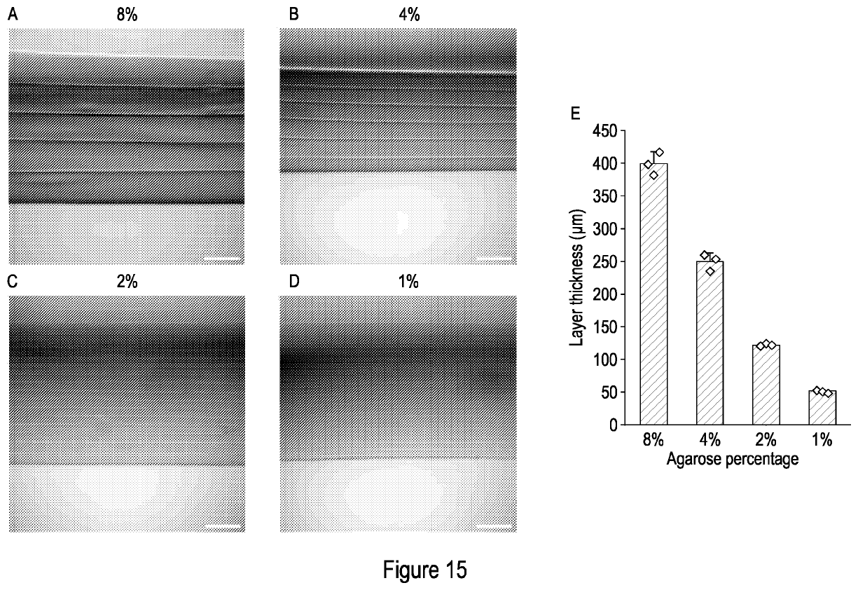

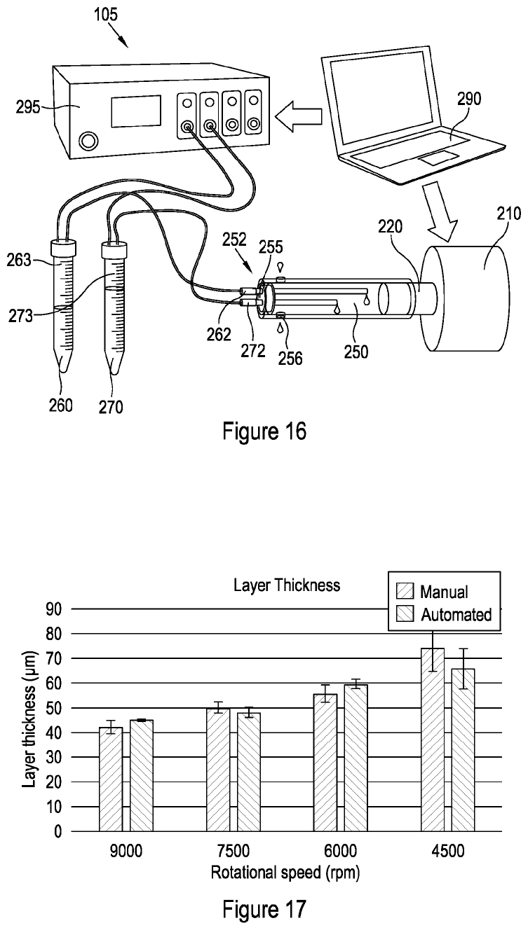

FIG. 7 shows a bright field microscopy image of a tube wall of one of the tubes of FIG. 6, showing discrete alginate layers, the bottom-right scale bar being 500 μm. FIG. 16 shows an apparatus for manufacturing a tubular biological structure, according to another embodiment. The apparatus 205 of FIG. 15 is generally similar to the apparatus 105 of FIGS. 1-5, like parts being denoted by like numerals, but incremented by “100”. However, in this embodiment, the apparatus 205 is automatically operated.

Thus, in this embodiment, a computer 290 controls:
the rotation, e.g. actuation, speed and duration, of the tubular member 250;
the operation of the feed delivery device 295, here comprising multiple circulation units (such as air pressure systems or a pumps), which control the delivery of biological material 260 stored in first container 263 via delivery device 262, and of crosslinking or gelling agent 270 stored in second container 273 via delivery device 272.

It will be appreciated that there may be provided a plurality of control units or computers, each configured to control a separate element of the apparatus, e.g. the actuation of the tubular member 250 and the operation of the feed delivery device 295. There may also be provided a plurality of containers or further containers to feed or deliver further materials.

In addition, in the embodiment of FIG. 16, the apparatus 205 does not comprise a compressible stopper 155 as in FIG. 1, but instead comprises a blocking element 255 which consists of a ridge or shoulder extending radially inwards at the second or distal end 252 and a tubular blocking element which engages with the ridge or shoulder and partially extends from the second or distal end 252 into the tubular member 250. Like the stopper 155 of FIG. 1, the blocking element 255 has an opening in it sentral region which allows the delivery devices 262,272 to extends into the tubular member 250 without interfering with the tubular member 250, even upon rotation.

### EXAMPLES

### Examples 1 and 2: Investigation of the Relationship Between Rotation Speed and Layer Thickness

### Example 1

A multi-layer tube was made according to the method described in FIGS. 1-5, using a 1% (w/v) alginate at various rotational speeds. For each stage, a volume between 30 μl and 100 μl was deposited inside the tube before rotation.

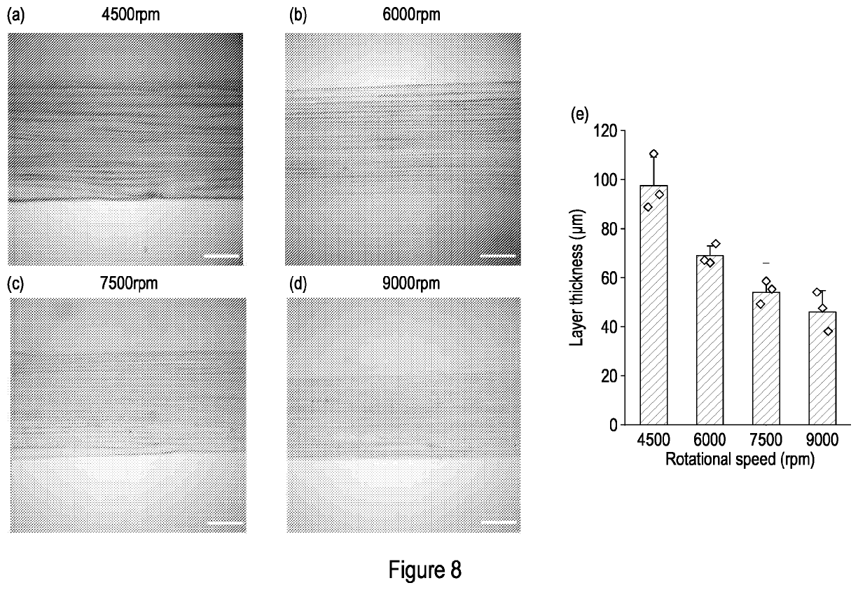

Layered tubes were made at speeds of 4500, 6000, 7500 and 9000 rpm. FIG. 8(a)-8(d) show bright field microscopy images of tube walls for each tube, respectively. FIG. 8(e) is a graph showing the relationship between rotation speed and layer thickness.

As shown, layer thicknesses were found to be a function of the speed of rotation. Analysis of the layer thicknesses revealed that increasing rotational speeds formed thinner layers, as illustrated in FIG. 8(e).

### Example 2

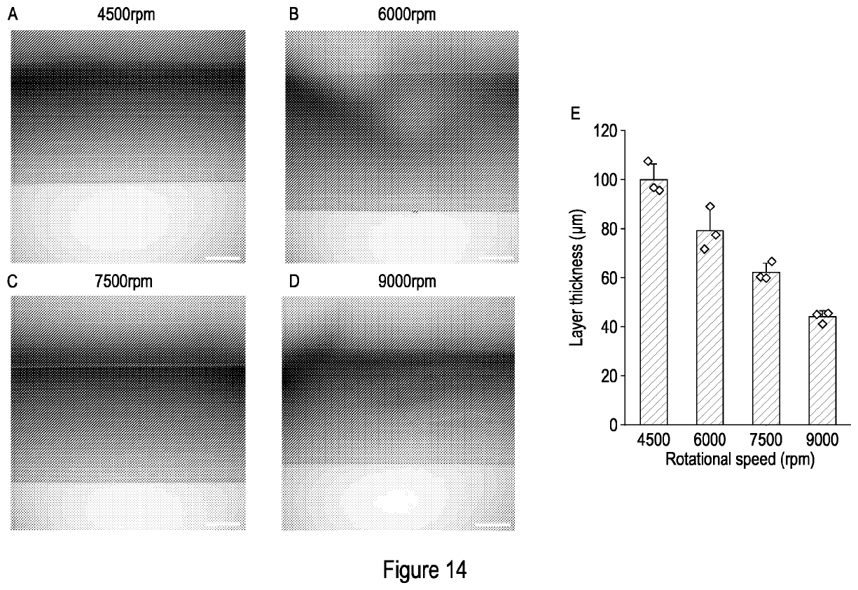

A similar experiment was conducted using a 1% (w/v) agarose at various rotational speeds. However, the preparation of an agarose-based structure did not require the addition of a crosslinker. For each stage, a volume between 30 μl and 100 μl was deposited inside the tube before rotation. Layered tubes were made at speeds of 4500, 6000, 7500 and 9000 rpm. FIG. 14(a)-14(d) show bright field microscopy images of tube walls for each tube, respectively. FIG. 14(e) is a graph showing the relationship between rotation speed and layer thickness.

As shown, layer thicknesses were found to be a function of the speed of rotation. Analysis of the layer thicknesses revealed that increasing rotational speeds formed thinner layers, as illustrated in FIG. 14(e).

### Examples 3 and 4: Investigation of the Relationship Between Concentration and Layer Thickness

### Example 3

A multi-layer tube was made according to the method described in FIGS. 1-5, using an (w/v) alginate solution at various concentrations, formed at a rotational speed of 9000 rpm.

Layered tubes were made using alginate concentrations of (w/v) (a) 4%, (b) 2%, (c) 1% and (d) 0.5%.

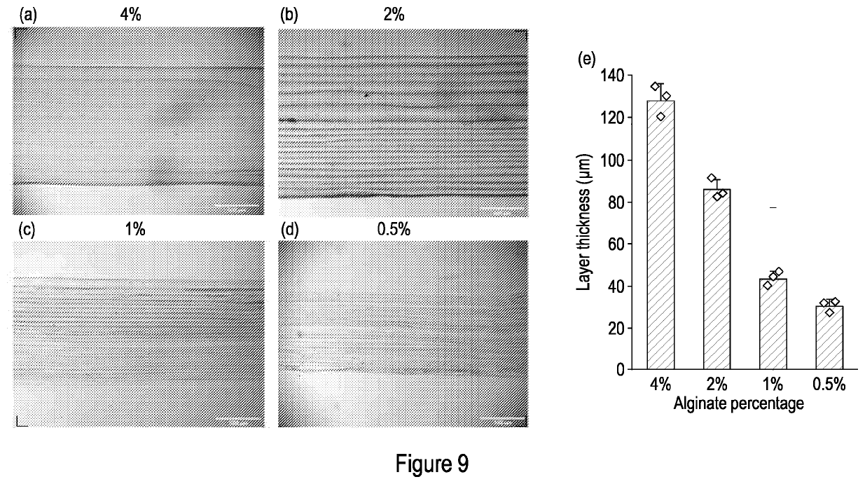

FIG. 9(a)-9(d) show bright field microscopy images of tube walls for each tube, respectively. FIG. 9(e) is a graph showing the relationship between alginate concentrations and layer thickness.

As shown, layer thicknesses were found to be a function of the alginate concentration. Analysis of the layer thicknesses revealed that increasing concentrations formed thicker layers, as illustrated in FIG. 9(e).

Without wishing to be bound by theory, it is believed that higher percentage concentration alginates are more viscous, establishing a positive correlation between layer thickness and viscosity. Higher viscosity materials are believed to require greater speeds and thus greater centrifugal forces to make the transition to rimming flow and thus reach a particular thickness.

When seeking to use low viscosity hydrogels, such as low concentration alginate or collagen, researchers often find that they are unable to form microscale features using traditional bioprinting methods. A compromise between degradation properties and printability is often needed. The present system is able to form a microscale multi-layered structure using low viscosity biological materials (such as alginate) by forming a stable layer of the material prior to addition of a gelation agent (when required).

### Example 4

A similar experiment was conducted using an agarose composition to form a tube at a rotational speed of 9000 rpm. However, the preparation of an agarose-based structure did not require the addition of a crosslinker.

Layered tubes were made using agarose concentrations of (w/v) (a) 8%, (b) 4%, (c) 2% and (d) 1%.

FIG. 15(a)-15(d) show bright field microscopy images of tube walls for each tube, respectively. FIG. 15(e) is a graph showing the relationship between agarose concentrations and layer thickness.

As shown above with alginate compositions, layer thicknesses with agarose were found to be a function of the agarose concentration. Analysis of the layer thicknesses revealed that increasing concentrations formed thicker layers, as illustrated in FIG. 15(e).

### Example 5: Investigation of the viability of live cells using the present method

A multi-layer tube was made according to the method described in FIGS. 1-5, using HEK cells encapsulated into 1% (w/v) alginate, formed at a rotational speed of 9000 rpm. In this example, the concentration of HEK cells in the biological material was 2.8×10⁶ cells/ml.

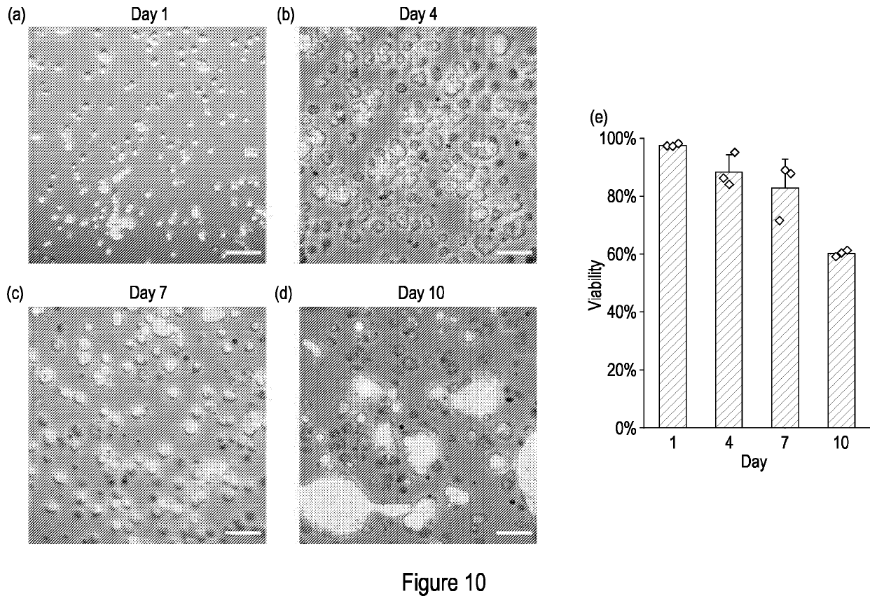

FIG. 10(a)-10(d) show combined bright field and fluorescence microscopy images of tube walls formed using such a material, showing live (green) and dead (red) cells at a) day 1, b) day 4, c) day 7 and d) day 10.

Cell viability data following layer encapsulation is shown in FIG. 10(e), with error bars being ±SD, n=3, and the white scale bars being 100 μm.

HEK cells encapsulated into 1% (w/v) alginate layers were shown to have a day 1 viability of 97.5%±0.4. The proportion of viable cells decreased in subsequent days to 60.4%±1.2 at day 10, as shown in FIG. 10(e). Without wishing to be bound by theory, the reduction in viability may be attributed to the formation of necrotic cores as cells proliferated and transitioned into larger spheroid bodies.

The data indicate that the overall forming protocol of trypsinisation, pelletization, suspension in alginate and the present process results in a high cell viability with similar percentages to those attained using extrusion bioprinting. Throughout the present process, cells are subjected to centrifugal forces arising from tube rotation and also shear forces from fluid flow. Simple centrifugal force calculations reveal that a single cell is subjected to forces an order of magnitude lower than standard centrifugation used during pelletization (1.55×10-11N vs 1.22×10-10N). Shear forces are also believed to have a detrimental impact on cell viability with Mironov et al. proposing that shear forces arising from cell transfer rather than centrifugal forces were responsible for cell death in their closed cylinder system. The present viability data demonstrates that shear forces are low enough for cell survival and indicate that cell encapsulation at higher rotational speeds than those presented here may be feasible.

### Example 6: Investigation of Cell Layer Positioning

A multi-layer tube was made according to the method described in FIGS. 1-5, using HEK293 cells encapsulated into 1% (w/v) alginate, formed at a rotational speed of 9000 rpm. The composition comprised high-density cell populations.

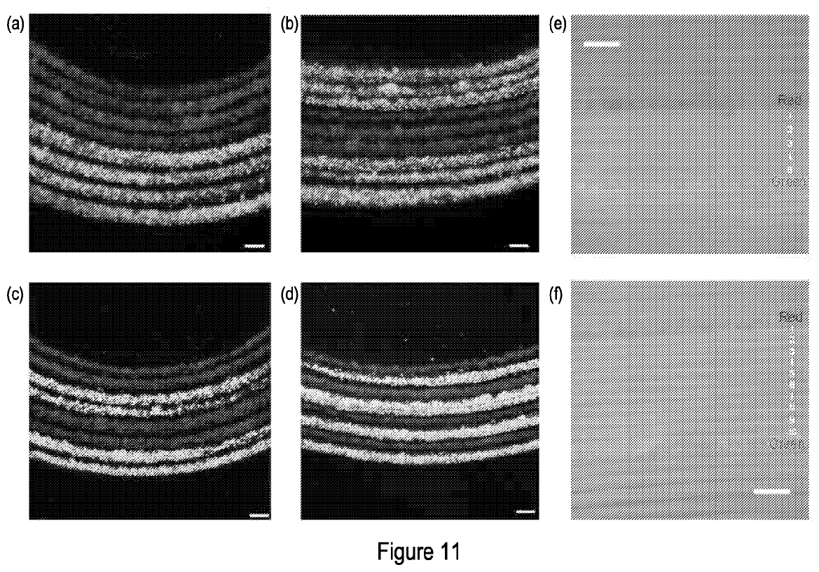

FIG. 11(a)-11(d) show combined bright field and fluorescence microscopy images of tube walls showing position of prelabelled red and green high density HEK293 cells in adjacent patterned layers formed using 1% w/v alginate. The cell concentration in the biological material used to makes these structures was 1.0×10⁸ cells/ml.

The labelled layers were assembled using varying patterns, as shown in FIG. 11(a)-11(d).

FIG. 11(e)-11(f) show combined bright field and fluorescence microscopy images of tube walls showing position of prelabelled red and green high density HEK293 cells layers formed using 1% w/v alginate with either with 5 (FIG. 11(e)) or 10 (FIG. 11(f)) acellular layers therebetween. The cell concentration in the biological material used to makes these structures was 2.7×10⁶ cells/ml.

The results confirm that each layer is formed individually and is discrete from its adjacent layers, allowing the potential formation of highly varied composite layered structures. High-density cell layers were observed to be thicker than acellular and low-density layers.

The results also demonstrate that the low volume of hydrogel needed for assembly enables high cell density encapsulation. In contrast, conventional tubular biofabrication technologies typically require large volumes for immersion, spraying or extrusion.

Stratified tissue varies considerably throughout mammalian anatomy, with multiple combinations of cell types, matrices and microlayer anatomy thicknesses. Biofabrication methods that are able to account for this variation through controlled layer deposition encapsulating a high density of cells can have a valuable role in the future development of highly representative 3D tissue. The precise spatial distancing of independent cell populations from one another may also have applications beyond tissue engineering, such as in the investigation of cell-to-cell signalling.

### Example 7: Formation of Vascular Collagen-Based Cell Structures

A multi-layer tube was made according to the method described in FIGS. 1-5, using hydrogel collagen and encapsulated human vascular smooth muscle cells (hSMCS), formed at a rotational speed of 4500 rpm, and carried out at 37° C. A 3-minute gelation time was applied for each collagen layer rather than the ionic agent addition for alginate.

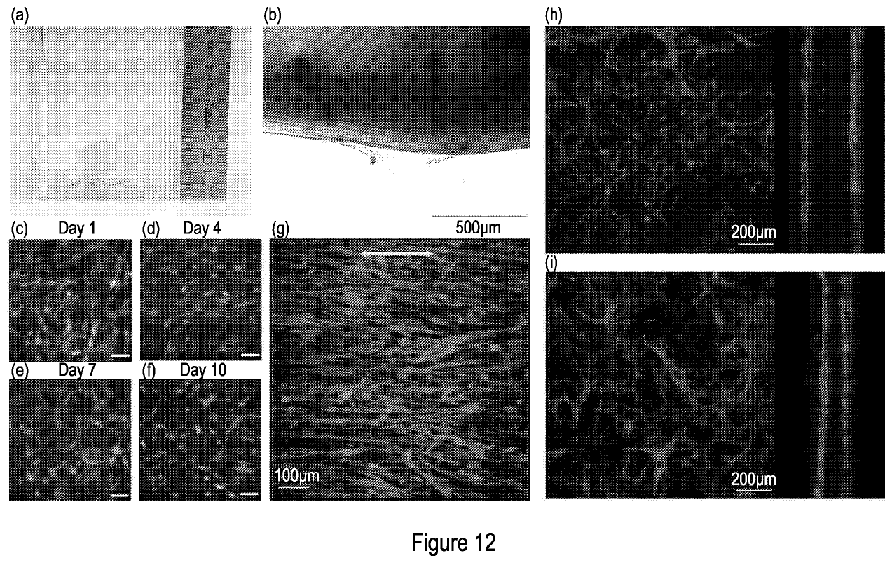

FIG. 12(a)-12(f) illustrate the investigation of such structures.

FIG. 12(a) shows a macroscale collagen tube architecture with an opaque white appearance, made by the present method.

Acellular tubes were also formed. FIG. 12(b) shows a microscale acellular collagen layer formation of the structure. This shows evidence of microscale layer formation when viewed using brightfield microscopy. However, visualisation of layers was more difficult in comparison to alginate tubes due to the opacity of the gelled collagen, with layers of ≈15 μm visible on the outer edge of the wall (FIG. 12(b)). The requirement to allow each layer 3 minutes to gel extends the biofabrication time, with 25 collagen layers taking approximately 1.5 hours.

FIG. 12(c) to 12(f) show the viability of hSMC cells in collagen using the present process, with live (green) and dead (red) cells at 1, 4, 7 and 10 days post biofabrication. The cell concentration in the biological material used to makes these structures was 7.4×10⁶ cells/ml. These images demonstrate that hSMCs added as a single layer amongst acellular layers had viability for up to 10 days post biofabrication. FIG. 12(g) shows hSMC orientation 4 days after stratification with yellow arrow indicating circumferential direction. The cell concentration in the biological material used to makes this structure was 0.6×10⁶ cells/ml. This visualisation revealed a network of hSMCs aligned in the circumferential direction. Such cellular orientation in microscale layers closely mimics the microscale anatomy observed in native vascular tissue. Cell orientation following encapsulation presents a challenge for tissue engineering strategies. Without wishing to be bound by theory, it is believed that the circumferentially directed flow of liquid phase collagen, caused by the rotation of the moulding tube, and the static tension induced by compaction onto the inner mandrel are likely to be contributing to the alignment of hSMCs. Tensile forces can be identified as a key contributor as hSMCs encapsulated in a single layer in the viability and positioning experiments did not exhibit any visible compaction forces on the macroscale tube and did not show any discernible alignment.

FIGS. 12(h) and 12(i) show confocal stacks showing hSMC positioning with 20 (h) and 10 (i) acellular layer spacing therebetween. F-actin visualisation via phalloidin staining and confocal microscopy revealed spread hSMCs in two distanced, independent, narrow cell-width microscale layers. The distance between cell layers in the 10 and 20 layer assemblies increased proportionately, with an average layer thickness of 12.5±1.7 μm. This distance closely matches published measurements of the thickness of the native Medial Lamellar Unit measured at 13.9±1.2 μm (rat) and 13.2 μm (human). The assembly of viable hSMCs into concentric, anatomically accurate layers represents an important progression for vascular biofabrication technologies. Bioprinting studies frequently only refer to the macrolayers of the tunica intima, media or adventitia, oversimplifying the complex microscale layered architecture within these structures, such as the Medial Lamellar Unit. The presented system allows encapsulating vascular cells in anatomically accurate concentric Medial Lamellar Units in the native ECM material of collagen.

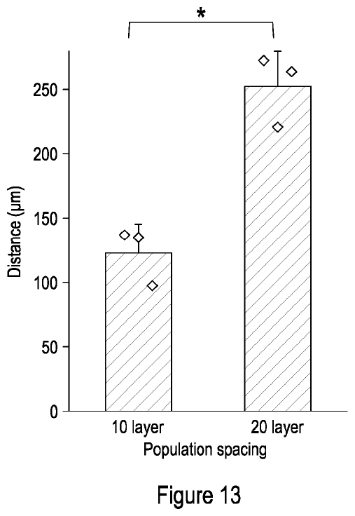

FIG. 13 shows distances between the human smooth muscle cell layers caused by a set number of acellular layers in between the 2 populations. These correspond to the double vertical lines in FIG. 12(h) (20 layers, hence a larger gap) and FIG. 12(i) (10 layers, hence a smaller gap).

### Example 8: Investigation of Automation of the Apparatus

Layer thickness and cell viability were investigated-in a similar manner to the experiments of FIG. 8 (example 1) and FIG. 10 (example 5), using the automated apparatus of FIG. 16.

FIG. 17 shows a comparison of the layer thickness obtained using a manual approach vs an automated approach. It can be seen that the automated approach yields similar results and therefore represents a reliable alternative.

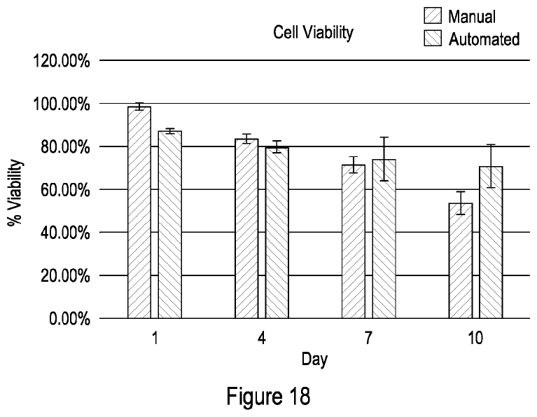

FIG. 18 shows a comparison of the cell viability measured using a manual approach vs an automated approach. It can be seen that the automated approach yields similar results and therefore represents a reliable alternative.

It will be understood that the present embodiments are provided by way of example only, and that various modifications can be made to the present embodiments without departing from the scope of the invention.

1. An apparatus for manufacturing a tubular biological structure, the apparatus comprising:
a tubular member configured to be rotated,
wherein the tubular member comprises a tubular wall extending between a first or proximal end and a second or distal end,
wherein the tubular member is configured to receive a biological material on an internal surface thereof, and
wherein the tubular member comprises at least one opening through the tubular wall configured to allow egress of excess biological material, in use.

2. An apparatus according to claim 1, wherein the apparatus comprises a rotatable support configured to be rotated, wherein the tubular member is configured to be mounted on or attached to the rotatable support.

3. An apparatus according to claim 1, wherein the tubular member defines an open tube having a first open end and a second open end.

4. An apparatus according to claim 1, wherein the apparatus comprises a motor capable of actuating rotation of the tubular member or of the rotatable support.

5. An apparatus according to claim 1, wherein the apparatus comprises a blocking element configured to prevent egress of the biological material, in use, at the second or distal end.

6. An apparatus according to claim 5, wherein the blocking element is provided at a second or distal end of the tubular member.

7. An apparatus according to claim 5, wherein the blocking element is configured to allow the delivery of the biological material within the tubular member on an internal surface of the tubular member.

8. An apparatus according to claim 5, wherein the blocking element comprises an opening therein.

9. An apparatus according to claim 1, wherein the at least one opening extends through a wall of the tubular member from an internal surface to an external surface thereof.

10. An apparatus according to claim 1, wherein the tubular member comprises a plurality of openings configured to allow egress of excess biological material, in use.

11. An apparatus according to claim 10, wherein the openings are circumferentially disposed on the tubular member.

12. An apparatus according to claim 10 wherein the openings are symmetrically disposed around a circumference of the tubular member.

13. An apparatus according to claim 1, wherein the biological material comprises a hydrogel and/or wherein the biological material comprises a cellular material.

14. (canceled)

15. An apparatus according to claim 1, further comprising a control unit configured to control the rotation of the tubular member.

16. An apparatus according to claim 15, wherein the control unit is configured to control the speed of rotation of the tubular member and/or of the rotatable support, wherein the control unit is configured to control and/or adjust the speed of rotation to a speed in the range of about 4000-10000 rpm.

17. (canceled)

18. A method of manufacturing a tubular biological structure, the method comprising:
providing an apparatus comprising a tubular member configured to be rotated, wherein the tubular member comprises a tubular wall extending from a first end and a second end, wherein the tubular member comprises at least one opening through the tubular wall configured to allow egress of excess biological material, in use;
feeding a first amount of a biological material on an internal surface of the tubular member; and
rotating the tubular member.

19. A method according to claim 18, the method further comprising adding or feeding a first amount of gelation/crosslinking agent inside the tubular member, and rotating the tubular member.

20. A method of manufacturing a tubular multi-layered biological structure, the method comprising:
(i) providing an apparatus comprising a tubular member configured to be rotated, wherein the tubular member comprises a tubular wall extending from a first end and a second end, wherein the tubular member comprises at least one opening through the tubular wall configured to allow egress of excess biological material, in use;
(ii) feeding a first amount of a biological material on an internal surface of the tubular member;
(iii) rotating the tubular member to form a first layer;
(iv) optionally adding or feeding a first amount of gelation/crosslinking agent inside the tubular member to cause gelling and/or crosslinking of the first layer; and rotating the tubular member;
(v) repeating steps (ii)-(vi) to form one or more additional layers.

21. A method according to claim 18, further comprising cutting the structure, so as to obtain a planar biological structure.

22. A biological structure obtained or obtainable by the method according to claim 18.
